(19) United States
(12) Patent Application Publication
(10) Pub. No.: US 2025/0226069 A1
(43) Pub. Date: Jul. 10, 2025

(54) SYSTEM FOR PROVIDING IMMUNE DATA TREATMENT, AND METHOD AND PROGRAM FOR PREDICTING IMMUNE STATUS DATA

(71) Applicant: Edgewater Corp., Setagaya-ku, Tokyo (JP)

(72) Inventor: Masahiko FUKUZAWA, Setagaya-ku,Tokyo (JP)

(21) Appl. No.: 18/850,810

(22) PCT Filed: Sep. 22, 2022
(86) PCT No.: PCT/JP2022/035389

§ 371 Date: Sep. 25, 2024

(51) Int. Cl. G16H 20/00; G16H 50/20

(52) U.S. Cl. CPC G16H 20/00; G16H 50/20

(57) ABSTRACT

The system for providing immune data treatment acquires immune data and treatment guideline data and then creates a learning dataset associated with the acquired immune data and treatment guideline data. The system learns the created learning data set and generates a learned model based on the learning result. New immune data of a new patient is acquired and the system determines a treatment guideline data part corresponding to the acquired new immune date by using the generated learned model. A treatment is extracted and included in the determined treatment guideline data part and is provided to a doctor or medical institution.

### CROSS-REFERENCE TO RELATED APPLICATIONS

This application is a national stage of International Application No. PCT/JP2022/035389, filed Sep. 22, 2022, which is hereby incorporated by reference in its entirety.

### TECHNICAL FIELD

The present invention relates to a system for providing an immune data treatment, and a method and a program for predicting immune status data.

### BACKGROUND

Generally, it is known that immunity is important in the incidence and recovery of Covid-19 and cancer (malignant neoplasms). Recently, due to the so-called coronavirus catastrophe the focus, on immunity has increased, and the demand for functional foods labelled as an immune functional food is also increasing. Therefore, it is expected that doctors should understand the patient's immune status and determines the best treatment for a patient based thereon.

As various treatments are established, it is extremely difficult to cover a array of treatments. Therefore, medical support technologies that present diagnostic and treatment proposals using AI, etc., to healthcare workers are attracting attention.

For example, the technology that generates descriptions of abnormalities in medical images from medical images and medical reports (treatment guidelines) and determines the order of treatments for patients (Patent Document 1-JP 2020-149682 A) is disclosed. The support system that provides treatment policies from breed information on target animals for regenerative medicine and treatment performance information for the breed is also disclosed (Patent Document 2-JP 2020-101843 A).

### SUMMARY

### The Technical Problem Solved by the Disclosure

However, the technologies described in Patent Documents 1 and 2 cannot associate and learn immune data with a treatment guideline, or cannot extract and provide a suitable treatment for a patient.

An objective of the present invention is to associate and learn immune data with a treatment guideline and extract and provide a suitable treatment for a patient.

### Solution for Solving the Technical Problem

### The Present Invention Provides the Following Solution.

A first aspect of the present invention provides a system for providing an immune data treatment, the system associating and learning immune data with a treatment guideline and extracting and providing a suitable treatment for a patient, comprising:
a first acquisition unit that acquires immune data and treatment guideline data;
a creation unit that creates a learning data set associated with the acquired immune data and treatment guideline data;
a learning unit that learns the created learning data set;
a generation unit that generates a learned model based on a learning result;
a second acquisition unit that acquires new immune data of a new patient;
a determination unit that determines a treatment guideline data part corresponding to the acquired new immune data by using the generated learned model;
an extraction unit that extracts a treatment included in the determined treatment guideline data part; and
a provision unit that provides the extracted treatment to a doctor or medical institution.

The first aspect of the present invention can create a learning data set associated with the acquired immune data and treatment guideline, learn the learning data set, generate a learned model, determine treatment guideline data part corresponding to a newly acquired immune data based on the generated leaned model, extract a treatment, and provide the treatment to a doctor or medical institution.

According to a second aspect of the present invention,
the system for providing an immune data treatment according to the first aspect of the present invention receives an advertisement from a company, determines an advertisement related to a treatment, and provides the treatment together with the determined advertisement to a doctor.

According to the second aspect of the present invention, the system for providing an immune data treatment according to the first aspect of the present invention can determine advertisement data related to treatment guideline data part corresponding to the acquired new immune data, extract the determined advertisement data, and provide the extracted advertisement data together with the extracted treatment to a doctor or medical institution.

According to a third aspect of the present invention, in the system for providing an immune data treatment according to the first aspect of the present invention,
the first acquisition unit further acquires health checkup data and lifestyle habit data;
the creation unit further creates a learning data set associated with the health checkup data and lifestyle habit data;
the second acquisition unit acquires new health checkup data and new lifestyle habit data of a new patient; and
the determination unit determines treatment guideline data part corresponding to the acquired new immune data, new health checkup data, and new lifestyle habit data by using the generated learned model.

According to the third aspect of the present invention, the system for providing an immune data treatment according to the first aspect of the present invention can create a learning data set associated with the acquired health checkup data, the lifestyle habit data, and the treatment guideline data, in addition to the immune data, learn the created learning data set, and generate a learned model, in order to improve the accuracy of treatment determination.

Furthermore, according to the third aspect of the present invention, the system for providing an immune data treatment according to the first aspect of the present invention can determine the treatment guideline data part corresponding to the acquired new immune data, new health checkup data, and new lifestyle habit data of a new patient, based on the learned model.

According to a fourth aspect of the present invention, in the system for providing an immune data treatment according to the first aspect of the present invention, the first acquisition unit further acquires treatment data from a medical institution;
the creation unit further creates a learning data set associated with the treatment data;
the second acquisition unit further acquires new treatment data of a new patient; and
the determination unit determines the treatment guideline data part corresponding to the acquired new immune data and new treatment data by using the generated learned model.

According to the fourth aspect of the present invention, the system for providing an immune data treatment according to the first aspect of the present invention can create a learning data set associated with the treatment data acquired from the medical institution and the treatment guideline data in addition to the immune data, learn the created learning data set, and generate a learned model, in order to improve the accuracy of treatment determination.

Furthermore, according to the fourth aspect of the present invention, the system for providing an immune data treatment according to the first aspect of the present can determine the treatment guideline data part corresponding to the acquired new immune data and new treatment data of a new patient, based on the learned model.

The first aspect of the present invention is a category of system, but also achieved in the categories of method and program, and achieves the structure, action, and effect in each category.

### Technical Effect

According to the present invention, the system for providing an immune data treatment, and a method and a program enables a doctor to determine an appropriate treatment for a patient by use of immune data.

### BRIEF DESCRIPTION OF THE DRAWINGS

FIG. 1 shows a basic overview of the system for providing an immune data treatment.

FIG. 2 shows a basic configuration of the system for providing an immune data treatment.

FIG. 3 is a flowchart showing the procedure of the learned model creation process performed by the computer of the system for providing an immune data treatment.

FIG. 4 is a flowchart showing the procedure of the treatment extraction and provision process performed by the computer of the system for providing an immune data treatment.

FIG. 5 is an example display screen of new immune data, which is displayed on a user terminal.

FIG. 6 is an example screen of treatment data and advertisement data created by the computer, which is displayed on a user.

FIG. 7 is a flowchart showing the procedure of the advertisement extraction and provision process performed by the computer of the system for providing an immune data treatment.

FIG. 8 is a flowchart showing the procedure of the learned model creation process utilizing health checkup data and lifestyle habit data, which is performed by the computer of the system for providing an immune data treatment.

FIG. 9 is a flowchart showing the procedure of the treatment extraction and provision process utilizing health checkup data and lifestyle habit data, which is performed by the computer of the system for providing an immune data treatment.

FIG. 10 is a flowchart showing the procedure of the learned model creation process utilizing treatment data, which is performed by the computer of the system for providing an immune data treatment.

FIG. 11 is a flowchart showing the procedure of the treatment extraction and provision process utilizing treatment data, which is performed by the computer of the system for providing an immune data treatment.

### DETAILED DESCRIPTION

The present invention will be more specifically described below with reference to the preferable embodiments. However, these are illustrative only, and the present disclosure is not limited thereto.

### Basic Overview/Basic Configuration

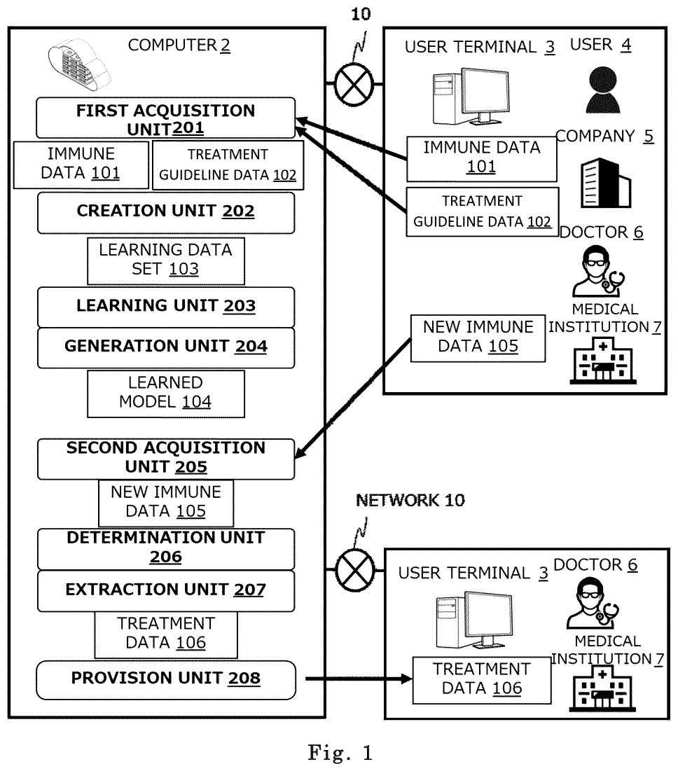

FIG. 1 is a diagram for explaining the overview of the system for providing immune data treatment 1. The basic overview of the system for providing immune data treatment 1 is explained below with reference to FIG. 1.

As shown in FIG. 1, the system for providing immune data treatment 1 is a computer system used for providing treatment utilizing immune data, including a computer 2 and a user terminal 3.

The computer 2 of the system for providing immune data treatment 1 is an on-premises server or computing system, or a cloud server or computing system. In this embodiment, the computer 2 is a cloud computing system.

The user terminal 3 of the system for providing immune data treatment 1 is a terminal for transmitting and receiving immune data 101, treatment guideline data 102, new immune data 105 of a new patient, etc., to and from the computer 2 by a user 4, a company 5, a doctor 6, or a medical institute 7. The user terminal 3 may be a personal computer or laptop, a mobile terminal such as a smartphone or tablet, or a wearable terminal such as a head-mounted display like smart glasses or a smartwatch.

The computer 2 of the system for providing immune data treatment 1 may be data-communicatively connected to the user terminal 3 through a network 10 such as a public line network and may transmit and receive necessary data and information.

The number of user terminals 3, users 4, third-party companies 5, doctors 6, and medical institutes 7 may be more than one.

The computer 2 of the system for providing immune data treatment 1 enables a doctor to determine a suitable treatment for a patient by performing the respective processes of:
a first acquisition unit 201 that acquires at least immune data 101 and treatment guideline data 102 from the user terminal 3;
   a learning data set 103 that creates a learning data set associated with the acquired immune data 101 and treatment guideline data 102;
   a learning unit 203 that learns the created learning data set 103;
   a generation unit 204 that generates a learned model 104 based on a learning result;
   a second acquisition unit 205 that acquires at least new immune data 105 of a new patient;
   a determination unit 206 that determines a treatment guideline data 102 part corresponding to the acquired new immune data 105 based on the learned model 104;
   an extraction unit 207 that extracts a treatment included in the determined treatment guideline data 102 part as treatment data 106; and
   a provision unit 208 that provides the extracted treatment data to a doctor 6 or medical institution 7.

The immune data 101 and new immune data 105 refer to data related to the immune status of a user or patient, for example, including at least immune data such as the amount of B cells, NK cells, T cells, total protein, or leukocytes.

The treatment guideline data 102 refers to data related to diseases and their treatment and coping, for example, including at least papers and clinical guidelines issued by medical societies, medical institutions, research institutions, companies, etc., based on scientific knowledge.

The treatment data 106 refers to data that includes at least a treatment part corresponding to the new immune data 105 included in the treatment guideline data 102.

The immune data 101, treatment guideline data 102, learning data set 103, learned model 104, new immune data 105, and treatment data 106 may each be stored either inside or outside the computer 2.

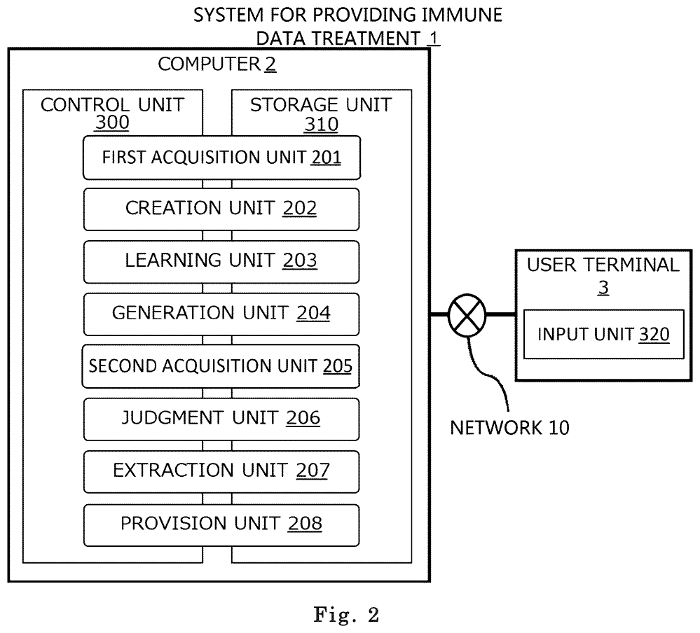

FIG. 2 is a diagram for explaining the basic configuration of the system for providing immune data treatment 1. The basic configuration of the system for providing immune data treatment 1 is explained below with reference to FIG. 2.

The computer 2 of the system for providing immune data treatment 1 is provided with a control unit 300 including a CPU (Central Processing Unit), GPU (Graphics Processing Unit), RAM (Random Access Memory), and ROM (Read Only Memory). The computer 2 is provided with at least a device such as a Wi-Fi® compatible device to enable communication with other terminals or devices. The computer 2 is provided with data storage unit such as a semiconductor memory, a recording medium, or a memory card as a storage unit 310. The storage unit may exist externally through network communication.

The computer 2 of the system for providing immune data treatment 1 is provided with the first acquisition unit 201, creation unit 202, learning unit 203, generation unit 204, second acquisition unit 205, determination unit 206, extraction unit 207, and provision unit 208 in addition to the above-mentioned control unit 300 and storage unit 310.

In the computer 2 of the system for providing immune data treatment 1, the control unit 300 achieves the first acquisition unit 201, creation unit 202, learning unit 203, generation unit 204, second acquisition unit 205, determination unit 206, extraction unit 207, and provision unit 208, by reading predetermined programs with cooperating with and storage unit 310.

The user terminal 3 is provided with an input unit 320 with the necessary function to operate the computer 2. The user terminal 3 can be provided with, for example, a liquid crystal display that achieves touch panel functionality, a keyboard, a mouse, a pen tablet, hardware buttons on the device, and/or a microphone for voice recognition, for input function. The present invention is not particularly limited in its functions by any input method.

The computer 2 of the system for providing immune data treatment 1 may be data-communicatively connected to the user terminal 3 through a network 10.

According to the system for providing immune data treatment 1, a doctor 6 can determine an appropriate treatment for a patient by use of immune data.

This is the basic overview and the basic configuration of the system for providing immune data treatment 1.

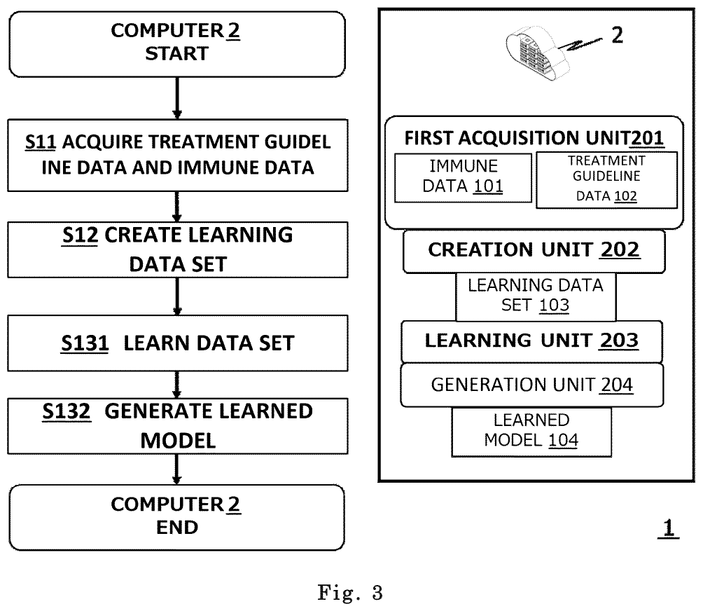

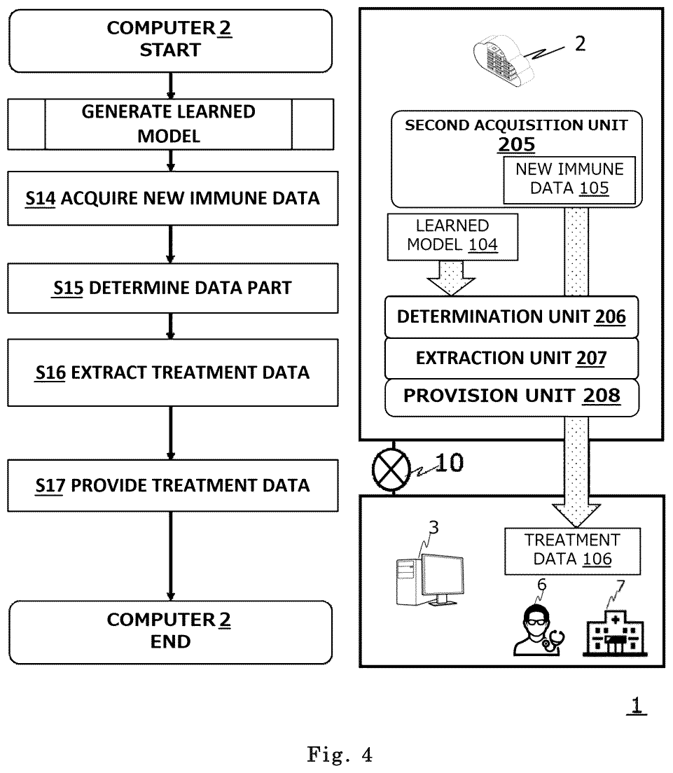

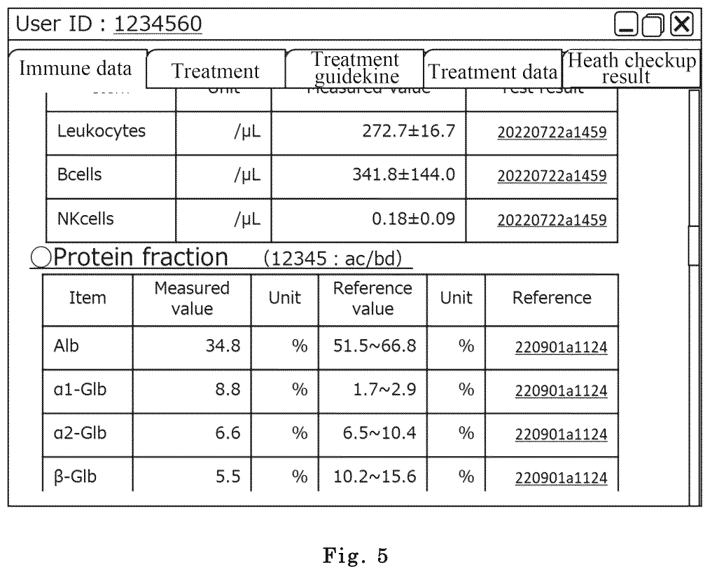

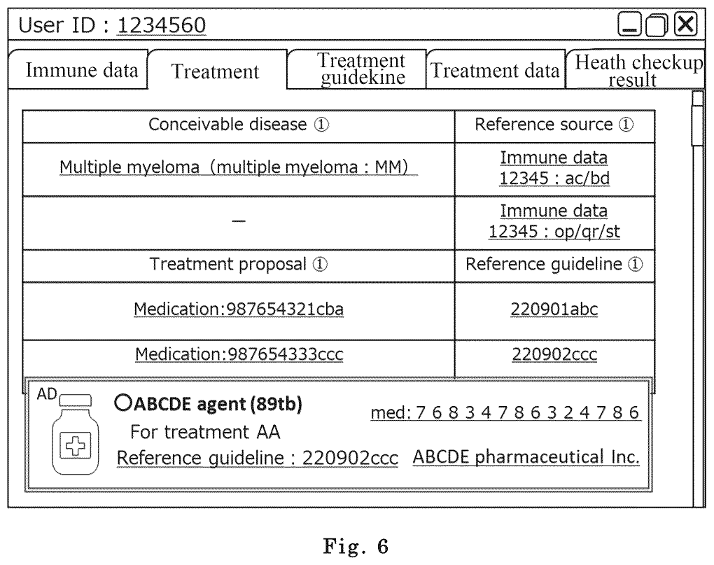

FIG. 3 is a diagram for explaining the learned model creation process performed by the computer 2 of the system for providing immune data treatment 1. FIG. 4 is a diagram for explaining the treatment extraction and provision process performed by the computer 2. FIG. 5 is an example display screen of the immune data 101 acquired by the computer 2, which is displayed on the user terminal 3. FIG. 6 is an example display screen of treatment data 106 extracted by the computer 2, which is displayed on the user terminal 3. The immune data treatment provision process performed by the computer 2 of the system for providing immune data treatment 1 is explained below with reference to FIGS. 3 to 6.

### Learned Model Creation Process

The first acquisition unit 201 of the computer 2 acquires at least the immune data 101 and treatment guideline data 102 (Step S11).

The immune data 101 is data related to the immune of a user or patient, for example, including at least immune information such as the amount of B cells, NK cells, T cells, total protein, leukocytes, albumin, or globulin. The treatment guideline data 102 refers to data related to diseases and their treatment, for example, including at least papers and clinical guidelines issued by medical societies, medical institutions, research institutions, companies, etc., based on scientific knowledge.

The format of the immune data 101 and treatment guideline data 102 includes all formats such as images, tables, numbers, and text, but is not limited thereto. The method of acquiring the immune data 101 and treatment guideline data 102 is not limited to the user terminal 3. These data may also be acquired from other terminal devices through a public line, etc. Furthermore, the acquisition timing of the immune data 101 and treatment guideline data 102 has no limitations.

The creation unit 202 of the computer 2 creates a learning data set 103 from the acquired immune data 101 and treatment guideline data 102 (Step S12). The created learning data set 103 is used as annotation data in the generation of the learned model 104, which will be described later. The annotation data is teacher data used to train a machine learning model, which is annotated as information related to the immune data to combine data with each other by attaching a meaning to data and associating data with each other.

The learning unit 203 of the computer 2 learns the created learning data set 103 as teacher data (Step S131), and the generation unit 204 generates the learned model 104 based on the learning result (Step S132).

The creation of the learned model 104, for example, is performed by applying annotation data related to the change in values involved in immunity to the immune data 101 from scientific findings such as abnormalities seen in the values of leukocytes, which are cells involved in immunity and abnormalities also seen in the values of y-globulin in protein fractions, characteristically seen in specific diseases.

The method of annotating data is not particularly limited and may be annotated by a manual method or by using a tagging automation tool such as an annotation tool.

The acquired immune data 101 and treatment guideline data 102, the created learning data set 103, and the generated learned model 104 may each be stored either inside or outside the computer 2.

Accordingly, the system for providing immune data treatment 1 can mechanically perform associations of a vast number of patterns by using the learned model from the acquired immune data 101 and treatment guideline data 102.

This is the learned model creation process executed by the system for providing immune data treatment 1.

### Treatment Extraction and Provision Process

FIG. 4 is a diagram for explaining the treatment extraction and provision process performed by the computer 2 of the system for providing immune data treatment 1. The treatment extraction and provision process performed by the computer 2 of the system for providing immune data treatment 1 is explained below with reference to FIG. 4.

The generation of the learned model is the same process as the above-mentioned learned model generation process, so its explanation is omitted.

The second acquisition unit 205 of the computer 2 acquires at least new immune data 105 that is new immune data of a new patient (Step S14).

As shown in FIG. 5, the new immune data 105 is data related to immunity, for example, including at least immune information such as the amount of B cells, NK cells, T cells, leukocytes, albumin, or globulin and may be from a different or the same user and patient as those who belong to the immune data 101 acquired in the above-mentioned learned model generation process.

The format of the new immune data 105 includes all formats such as images, tables, numbers, and text, but is not limited thereto. The timing of acquiring the new immune data 105 has no limitations.

The determination unit 206 of the computer 2 determines the treatment guideline data 102 part corresponding to the acquired new immune data 105, based on the learned model 104 generated by the above-mentioned learned model generation process (Step S15).

The treatment guideline data 102 part that the determination unit 206 of the computer 2 determines, for example, indicates a part that includes at least one or more treatments corresponding to the new immune data 105 based on the learned model 104 that include considered diseases, details of the disease, preventions for the disease, treatments and symptomatic treatments for the disease, and that includes information on medical devices and therapeutic medicines related to the treatments.

The extraction unit 207 of the computer 2 extracts the treatment included in the part determined by the determination unit 206 as treatment data 106 (Step S16).

As shown in FIG. 6, the extracted treatment data 106, for example, indicates the treatment including the treatments and symptomatic treatments that are related to the disease contained in the treatment guideline data 102 part that the determination unit 206 of the computer 2 has determined.

The provision unit 208 of the computer 2 provides the extracted treatment data 106 to a doctor 6 or medical institution 7 (Step S16).

The method of providing the treatment data 106 by the provision unit 208 has no limitations and may be provided via the user terminal 3 that is communicatively connected via the network 10.

The acquired new immune data 105 and the extracted treatment data 106 may each be stored inside or outside the computer 2.

Accordingly, the system for providing immune data treatment 1 enables a doctor 6 to determine the treatment suitable for a patient by use of the immune data, by extracting and providing treatment data 106 from the acquired new immune data 105 by using the learned model 104. This is the treatment extraction and provision process executed by the system for providing immune data treatment 1.

### Advertisement Extraction and Provision Process

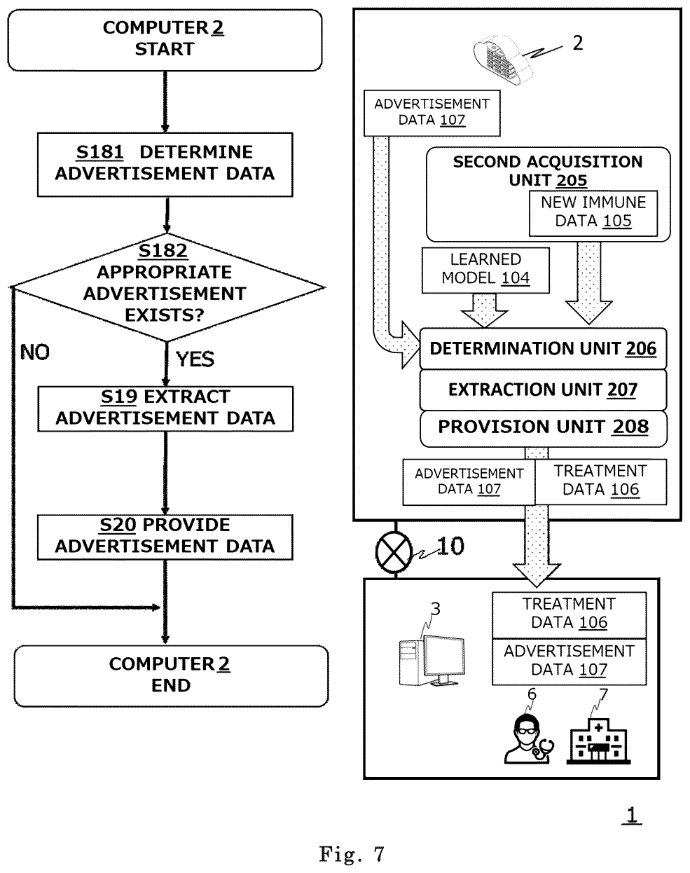

FIG. 7 is a diagram for explaining the advertisement extraction and provision process performed by the computer 2 of the system for providing immune data treatment 1. The advertisement extraction and provision process performed by the computer of the system for providing immune data treatment 1 is explained below with reference to FIG. 7

The advertisement extraction and provision processing performed by the computer 2 of the system for providing immune data treatment 1 is performed in accordance with the above-mentioned treatment extraction and provision process, as shown in FIG. 7.

If the determination unit 206 of the computer 2 determines the treatment guideline data 102 part corresponding to the acquired new immune data 105, based on the learned model 104 generated by the learned model generation process, the determination unit 206 determines one or more advertisement data 107 related to the treatment guideline data 102 part corresponding to the determined new immune data 105 (Step S181).

The advertisement data 107 refers to one or more advertisements issued by a company 5, etc., for example, advertisements for reagents such as test agents, pharmaceuticals such as therapeutic medicines, medical devices, or software related to medical care.

The method of acquiring the advertisement data 107 has no limitations. The acquired advertisement data 107 may be stored inside or outside the computer 2.

If there is advertisement data corresponding to the treatment guideline data 102 part determined by the determination unit 206 (Step S182 YES), the extraction unit 207 of the computer 2 extracts one or more determined advertisement data 107 (Step S19).

If there is no advertisement data corresponding to the treatment guideline data 102 part determined by the determination unit 206 (Step S182 NO), the extraction unit 207 of the computer 2 extracts no advertisement data 107.

The provision unit 208 of the computer 2 provides the one or more extracted advertisement data 107 to a doctor 6 or a medical institution 7 together with the treatment data 106 extracted in the above-mentioned treatment extraction and provision process (Step S20).

The advertisement data 107 provided together with the treatment data 106 is displayed as shown in FIG. 6.

Accordingly, the system for providing immune data treatment 1 can provide only advertisements related to the treatment by determining and extracting the advertisement data 107 corresponding to the extracted treatment data 106, and providing the advertisement data 107 together with the treatment data 106 to a doctor 6 and a medical institution 7.

### This is the Advertisement Extraction and Provision Process Executed by the System for Providing an Immune Data Treatment 1

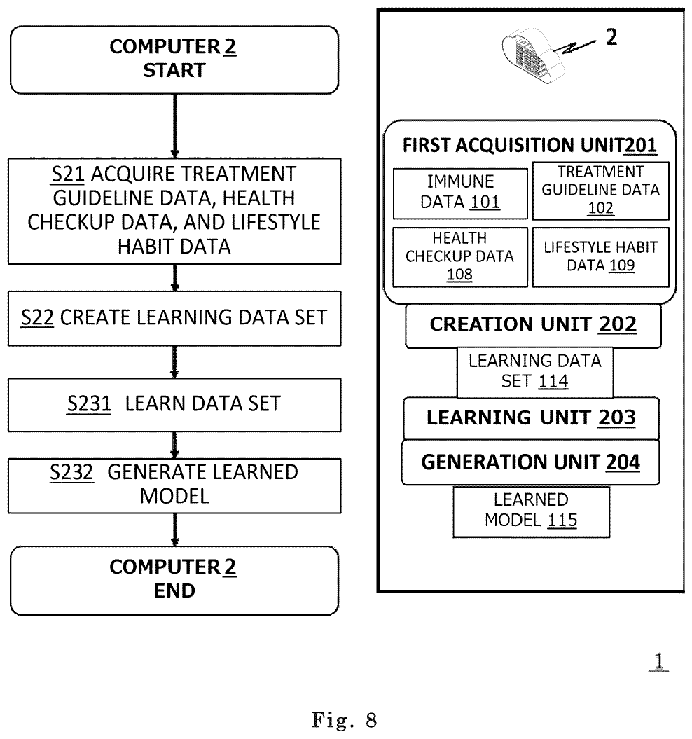

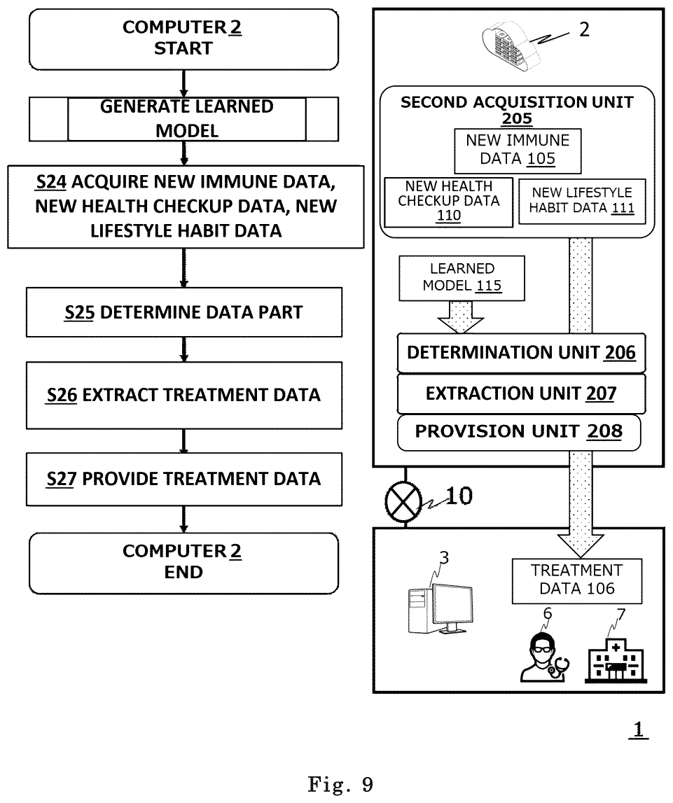

FIG. 8 is a diagram for explaining the procedure of the learned model creation process utilizing health checkup data and lifestyle habit data, which is performed by the computer 2 of the system for providing an immune data treatment 1. FIG. 9 is a diagram for explaining the procedure of the treatment extraction and provision process utilizing health checkup data and lifestyle habit data, which is performed by the computer 2. The immune data treatment provision process performed by the computer 2 of the system for providing immune data treatment 1 is explained below with reference to FIGS. 8 and 9.

### Learned Model Creation Process Utilizing Health Checkup Data and Lifestyle Habit Data

The first acquisition unit 201 of the computer 2 acquires at least the immune data 101, treatment guideline data 102, health checkup data 108, and lifestyle habit data 109 (Step S21).

The health checkup data 108 refers to data that includes at least the result of so-called health checkup, for example, school health checkup, workplace health checkup, specified checkup, and general checkup, received by the above-mentioned user or patient. The lifestyle habit data 109 includes at least data related to life style such as sleep duration over a certain period, sleep depth, or wake-up frequency during sleep; the number of steps, exercise frequency, or exercise duration; smoking frequency, drinking frequency, or the amount of alcohol consumed; active hours or sleeping hours; working hours or work content; or dietary status data such as meal content, snack frequency, calorie intake, or nutritional balance of the user or patient.

The format of the immune data 101, treatment guideline data 102, health checkup data 108, and lifestyle habit data 109 includes all formats such as images, tables, numbers, and text, but is not limited thereto. The method of acquiring the immune data 101, treatment guideline data 102, health checkup data 108, and lifestyle habit data 109 is not limited to the user terminal 3. These data may also be acquired from other terminal devices through a public line, etc. Furthermore, the acquisition timing of the immune data 101, treatment guideline data 102, health checkup data 108, and lifestyle habit data 109 has no limitations.

The creation unit 202 of the computer 2 creates a learning data set 114 from the acquired immune data 101, treatment guideline data 102, health checkup data 108, and lifestyle habit data 109 (Step S22). The created learning data set 114 is used as annotation data in the generation of the learned model 115, which will be described later. The annotation data is teacher data used to train a machine learning model, which is annotated as information related to the immune data, health checkup data, or lifestyle habit data to combine data with each other by attaching a meaning to data and associating data with each other.

The method of annotating data is not particularly limited and may be annotated by a manual method or by using a tagging automation tool such as an annotation tool.

The learning unit 203 of the computer 2 learns the created learning data set 114 as teacher data (Step S231), and the generation unit 204 generates the learned model 115 based on the learning result (Step S232).

The acquired immune data 101, treatment guideline data 102, health checkup data 108, and lifestyle habit data 109, the created learning data set 114, and the generated learned model 115 may each be stored either inside or outside the computer 2.

Accordingly, the system for providing immune data treatment 1 can mechanically perform associations of a vast number of patterns by using the learned model from the acquired immune data 101, treatment guideline data 102, health checkup data 108, and lifestyle habit data 109. This is the learned model creation process utilizing health checkup data and lifestyle habit data executed by the system for providing an immune data treatment 1.

### Treatment Extraction and Provision Process Utilizing Health Checkup Data and Lifestyle Habit Data

FIG. 9 is a flowchart showing the procedure of the treatment extraction and provision process utilizing health checkup data and lifestyle habit data, which is performed by the computer 2 of the system for providing an immune data treatment 1. The treatment extraction and provision process performed by the computer 2 of the system for providing immune data treatment 1 is explained below with reference to FIG. 9.

The generation of the learned model is the same process as the above-mentioned learned model creation process utilizing health checkup data and lifestyle habit data, so its explanation is omitted.

The second acquisition unit 205 of the computer 2 acquires at least new immune data 105 that is new immune data of a new patient, and also acquires at least new treatment data 113 (Step S24).

The new immune data 105 and new treatment data 113 refer to data related to immune data 101, health checkup data 108, and lifestyle data 109 These data may be different from or same as the immune data 101, health checkup data 108, and lifestyle data 109 acquired through the above-mentioned learned model creation process utilizing health checkup data and lifestyle habit data, which may belong to the user and/or patient.

The format of the new immune data 105 and new treatment data 113 includes all formats such as images, tables, numbers, and text, but is not limited thereto. The timing of acquiring the new immune data 105 and new treatment data 113 has no limitations.

The determination unit 206 of the computer 2 determines the treatment guideline data 102 part corresponding to the acquired new immune data 105 and new treatment data 113, based on the learned model 115 generated by the above-mentioned learned model generation process (Step S25).

The treatment guideline data 102 part that the determination unit 206 of the computer 2 determines, for example, indicates a part that includes at least one or more treatments corresponding to each of the new immune data 105 and the new treatment data 113 based on the learned model 115 that include considered diseases, details of the disease, preventions for the disease, treatments and symptomatic treatments for the disease, and that includes information on medical devices and therapeutic medicines related to the treatments.

The extraction unit 207 of the computer 2 extracts the treatment included in the part determined by the determination unit 206 as treatment data 106 (Step S26).

The extracted treatment data 106, for example, indicates the treatment including the treatments and symptomatic treatments that are related to the disease contained in the treatment guideline data 102 part that the determination unit 206 of the computer 2 has determined.

The provision unit 208 of the computer 2 provides the extracted treatment data 106 to a doctor 6 or medical institution 7 (Step S27).

The method of providing the treatment data 106 by the provision unit 208 has no limitations and may be provided via the user terminal 3 that is communicatively connected via the network 10.

The acquired new immune data 105 and the extracted treatment data 106 may each be stored inside or outside the computer 2.

Accordingly, the system for providing immune data treatment 1 enables a doctor 6 to more accurately determine the treatment suitable for a patient by use of the immune data, by extracting and providing treatment data 106 from the acquired new immune data 105 and new treatment data 113 by using the learned model 115. This is the treatment extraction and provision process utilizing health checkup data and lifestyle habit data executed by the system for providing immune data treatment 1.

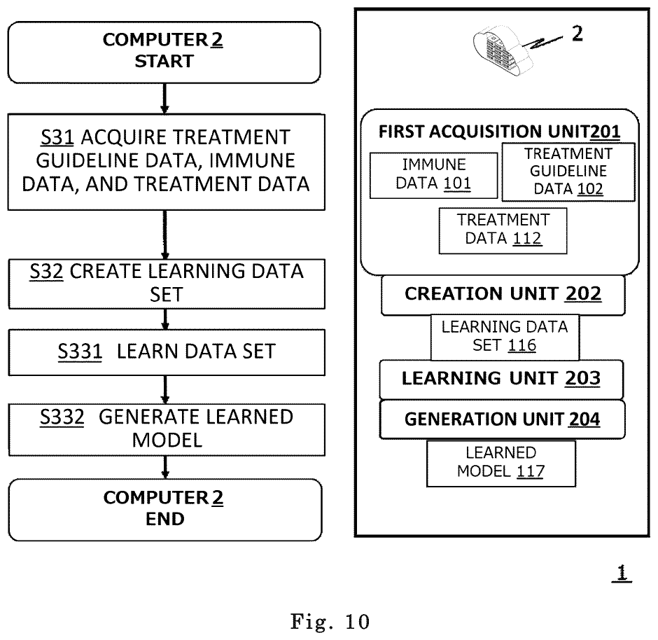

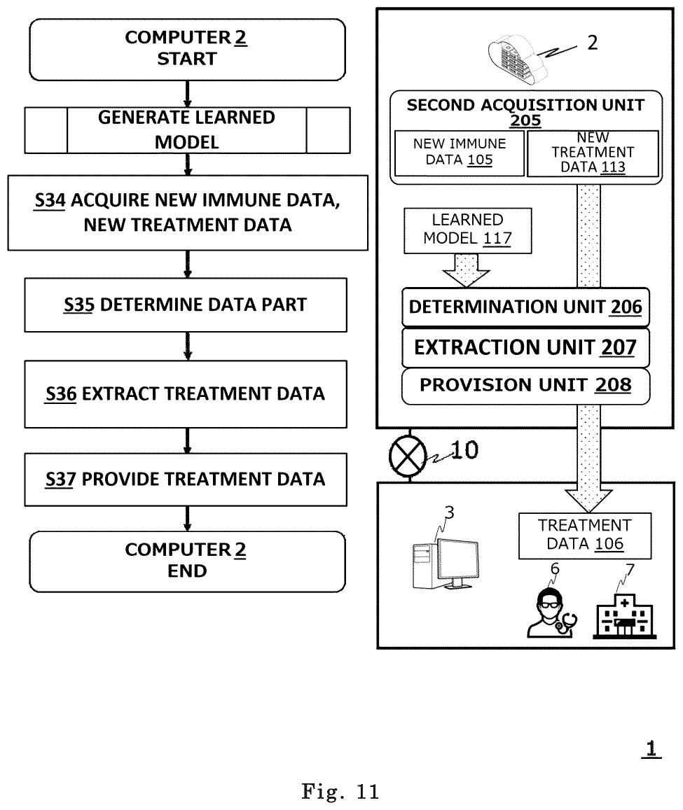

FIG. 10 is a diagram for explaining learned model creation process utilizing treatment data, which is performed by the computer 2 of the system for providing immune data treatment 1. FIG. 11 is a diagram for explaining the treatment data utilization extraction and provision process performed by the computer 2. The immune data treatment provision process performed by the computer 2 of the system for providing immune data treatment 1 is explained below with reference to FIGS. 10 and 11.

### Learned Model Creation Process Utilizing Treatment Data

The first acquisition unit 201 of the computer 2 acquires at least the immune data 101, treatment guideline data 102, and treatment data 112 (Step S31).

The treatment data 112 refers to data that includes at least information from the so-called medical records, including, for example, the examination progress, surgical records, test records, nursing records, and medical history of the above-mentioned user or patient. This data is acquired from a medical institution.

The format of the immune data 101, treatment guideline data 102, and treatment data 112 includes all formats such as images, tables, numbers, and text, but is not limited thereto. The method of acquiring the immune data 101, treatment guideline data 102, and treatment data 112 is not limited to the user terminal 3. These data may also be acquired from other terminal devices through a public line, etc. Furthermore, the acquisition timing of the immune data 101, treatment guideline data 102, and treatment data 112 has no limitations.

The creation unit 202 of the computer 2 creates a learning data set 116 from the acquired immune data 101, treatment guideline data 102, and treatment data 112 (Step S32). The created learning data set 116 is used as annotation data in the generation of the learned model 117, which will be described later. The annotation data is teacher data used to train a machine learning model, which is annotated as information related to the immune data or treatment data to combine data with each other by attaching a meaning to data and associating data with each other.

The method of annotating data is not particularly limited and may be annotated by a manual method or by using a tagging automation tool such as an annotation tool.

The learning unit 203 of the computer 2 learns the created learning data set 116 as teacher data (Step S231), and the generation unit 204 generates the learned model 117 based on the learning result (Step S232).

The acquired immune data 101, treatment guideline data 102, and treatment data 112, the created learning data set 116, and the generated learned model 117 may each be stored either inside or outside the computer 2.

Accordingly, the system for providing immune data treatment 1 can mechanically perform associations of a vast number of patterns by using the learned data from the acquired immune data 101, treatment guideline data 102, and treatment data 112.

This is the learned model creation process utilizing treatment data executed by the system for providing immune data treatment 1.

### Treatment Extraction and Provision Process

FIG. 11 is a diagram for explaining the treatment data utilization extraction and provision process performed by the computer 2 of the system for providing immune data treatment 1. The treatment extraction and provision process performed by the computer 2 of the system for providing immune data treatment 1 is explained below with reference to FIG. 11.

The generation of the learned model is the same process as the above-mentioned treatment utilization learned treatment data model generation process, so its explanation is omitted.

The second acquisition unit 205 of the computer 2 acquires at least new immune data 105 that is new immune data 105 of a new patient, and also acquires new treatment data 113 (Step S34).

The second acquisition unit 205 of the computer 2 acquires the new treatment data 113 from a medical institution 7.

The new immune data 105 and new treatment data 113 refer to data related to immune data 101 and treatment data 112. These data may be different from or same as the immune data 101 and treatment data 112 acquired through the above-mentioned learned model creation process utilizing treatment data, which may belong to the user and/or patient.

The format of the new immune data 105 and new treatment data 113 includes all formats such as images, tables, numbers, and text, but is not limited thereto. The timing of acquiring the new immune data 105 and new treatment data 113 has no limitations.

The determination unit 206 of the computer 2 determines the treatment guideline data 102 part corresponding to the acquired new immune data 105 and new treatment data 113, based on the learned model 117 generated by the above-mentioned learned model generation process (Step S35).

The treatment guideline data 102 part that the determination unit 206 of the computer 2 determines, for example, indicates a part that includes at least one or more treatments corresponding to each of the new immune data 105 and the new treatment data 113 based on the learned model 117 that include considered diseases, details of the disease, preventions for the disease, treatments and symptomatic treatments for the disease, and that includes information on medical devices and therapeutic medicines related to the treatments.

The extraction unit 207 of the computer 2 extracts the treatment included in the part determined by the determination unit 206 as treatment data 106 (Step S36).

The extracted treatment data 106, for example, indicates the treatment including the treatments and symptomatic treatments that are related to the disease contained in the treatment guideline data 102 part that the determination unit 206 of the computer 2 has determined.

The provision unit 208 of the computer 2 provides the extracted treatment data 106 to a doctor 6 or medical institution 7 (Step S37).

The method of providing the treatment data 106 by the provision unit 208 has no limitations and may be provided via the user terminal 3 that is communicatively connected via the network 10.

The acquired new immune data 105 and the extracted treatment data 106 may each be stored inside or outside the computer 2.

Accordingly, the system for providing immune data treatment 1 enables a doctor 6 to more accurately determine the treatment suitable for a patient by use of the immune data, by extracting and providing treatment data 106 from the acquired new immune data 105 and new treatment data 113 by using the learned model 117.

This is the treatment extraction and provision process executed by the system for providing immune data treatment 1.

The computer (including CPU, an information processor, and various terminals) reads and executes a predetermined program to achieve the above-mentioned means and functions. For example, the program may be provided by one or more computers (SaaS: Software as a Service) through a network or a cloud service. The program may be provided in a form recorded in a computer-readable recording medium, for example. In this case, the computer reads a program from the recording medium, and forwards it to an internal or external storage, records it in the storage, and executes it. The program may be previously recorded in a storage (recording medium) and provided from the storage to the computer through a communication line.

The embodiments of the present invention are described above, but the present invention is not limited thereto. Moreover, the effects described in the embodiments of the present invention are only the most suitable ones produced from the present invention. The effects of the present invention are not limited to those described in the embodiments of the present invention.

### DESCRIPTION OF REFERENCE NUMERALS

1: System for providing immune data treatment, 2: Computer, 3: User terminal, 4: User, 5: Company, 6: Doctor, 7: Medical institution, 10: Network

1. A system for providing an immune data treatment, the system associating and learning immune data with a treatment guideline and extracting and providing a suitable treatment for a patient, comprising:
a first acquisition unit that acquires immune data and treatment guideline data;
a creation unit that creates a learning data set associated with the acquired immune data and treatment guideline data;
a learning unit that learns the created learning data set;
a generation unit that generates a learned model based on a learning result;
a second acquisition unit that acquires new immune data of a new patient;
a determination unit that determines a treatment guideline data part corresponding to the acquired new immune data by using the generated learned model;
an extraction unit that extracts a treatment included in the determined treatment guideline data part; and
a provision unit that provides the extracted treatment to a doctor or medical institution.

2. The system for providing an immune data treatment according to claim 1, wherein the system receives an advertisement from a company, determines an advertisement related to a treatment, and provides the treatment together with the determined advertisement to a doctor.

3. The system for providing an immune data treatment according to claim 1, wherein
the first acquisition unit further acquires health checkup data and lifestyle habit data;
the creation unit further creates a learning data set associated with the health checkup data and lifestyle habit data;
the second acquisition unit acquires new health checkup data and new lifestyle habit data of a new patient; and
the determination unit determines treatment guideline data part corresponding to the acquired new immune data, new health checkup data, and new lifestyle habit data by using the generated learned model.

4. The system for providing an immune data treatment according to claim 1, wherein
the first acquisition unit further acquires treatment data from a medical institution;
the creation unit further creates a learning data set associated with the treatment data;
the second acquisition unit further acquires new treatment data of a new patient; and
the determination unit determines the treatment guideline data part corresponding to the acquired new immune data and new treatment data by using the generated learned model.

5. A method for providing an immune data treatment, the system associating and learning immune data with a treatment guideline and extracting and providing a suitable treatment for a patient, comprising the steps of:
acquiring immune data and treatment guideline data;
creating a learning data set associated with the acquired immune data and treatment guideline data;
learning the created learning data set;
generating a learned model based on a learning result;
acquiring new immune data of a new patient;
judging a treatment guideline data part corresponding to the acquired new immune data by using the generated learned model;
extracting a treatment included in the determined treatment guideline data part; and
providing the extracted treatment to a doctor or medical institution.

6. A non-transitory computer-readable storage medium on which a program is stored, wherein the program causes a computer system for providing an immune data treatment, and wherein the system associates and learns immune data with a treatment guideline and extracts and provides a suitable treatment for a patient to execute steps of:
acquiring immune data and treatment guideline data;
creating a learning data set associated with the acquired immune data and treatment guideline data;
learning the created learning data set;
generating a learned model based on a learning result;
acquiring new immune data of a new patient;
judging a treatment guideline data part corresponding to the acquired new immune data by using the generated learned model;
extracting a treatment included in the determined treatment guideline data part; and
providing the extracted treatment to a doctor or medical institution.
